(19) United States
(12) Patent Application Publication
(10) Pub. No.: US 2026/0196066 A1
(43) Pub. Date: Jul. 9, 2026

(54) METHOD AND APPARATUS FOR MEASURING MEAT TASTE AND TENDERNESS USING AN ARTIFICIAL INTELLIGENCE MODEL

(71) Applicant: DEEPLANT, INC., Goyang-si (KR)

(72) Inventor: Chul-beom KIM, Goyangsi (KR)

(21) Appl. No.: 19/555,247

(22) Filed: Mar. 3, 2026

Related U.S. Application Data
(63) Continuation of application No. PCT/KR2024/013014, filed on Aug. 30, 2024.

(30) Foreign Application Priority Data
Sep. 5, 2023 (KR) 10-2023-0117323

(51) Int. Cl. G06V 20/68; G06T 7/40; G06T 7/90; G06V 10/774

(52) U.S. Cl. CPC G06V 20/68; G06T 7/40; G06T 7/90; G06V 10/774

(57) ABSTRACT

An embodiment relates to a method for measuring meat taste and tenderness using an artificial intelligence model. The method includes generating a training data set including at least one training meat image and a sensory evaluation table matched to the training meat image, and generating an artificial intelligence model trained to measure taste and tenderness of meat included in an input meat image based on the training data set. The artificial intelligence model is trained using meat images classified by taste and tenderness to measure meat taste and tenderness in a consistent and accurate manner, and performs the measurement through artificial intelligence-based processing. In addition, criteria for meat grade evaluation are clarified, and information indicating which portions play a determining role in grade evaluation is provided.

### CROSS-REFERENCE TO RELATED APPLICATION

This application is a continuation of PCT Patent Application No. PCT/KR2024/013014 filed on August 30, 2024, which claims priority to Korean Patent Application No. 10-2023-0117323, filed on September 5, 2023, in the Korean Intellectual Property Office, the disclosure of which is incorporated by reference herein in its entirety.

### BACKGROUND

### [Technical Field]

The present invention relates to a method and an apparatus for measuring meat taste and tenderness using an artificial intelligence model, and more particularly, to a technology for analyzing a meat image using an artificial intelligence model and measuring the taste and tenderness of meat included in the image.

### [Background Art]

Globally, meat consumption is continuously increasing. As of 2021, chicken ranked first in global meat consumption, followed by pork and beef.

As of 2021, pork consumption was 32.3 kg, and domestic beef consumption was 12.4 kg per capita. Recent consumers can obtain information about meat through various media, and comprehensively consider such information to purchase higher-quality meat. This is because differences in taste and price according to meat grade are significant.

In purchasing meat, taste can be considered as one of the consideration factors including price, origin, and grade. Factors determining the taste of meat may include marbling, meat color, juiciness, and texture.

Conventionally, the taste and tenderness of meat were measured by experts, and consumers purchased meat based on the taste and tenderness measured by the experts.

However, evaluations of taste and tenderness may differ depending on the expert, because criteria such as marbling, meat color, texture, juiciness, and preference, which are major items for measuring the taste and tenderness of meat, vary depending on the evaluating expert. For this reason, consistency in the taste and tenderness of meat has been reduced.

Accordingly, there is an increasing need for a method and an apparatus for measuring meat taste and tenderness using artificial intelligence, which analyze a single meat image based on data possessed by artificial intelligence trained with a plurality of meat images classified by taste and tenderness, and measure taste and tenderness rapidly and consistently.

### SUMMARY

### [Problem to be Solved]

The present invention is intended to solve the problems of the conventional art described above, and relates to a method and an apparatus for determining a meat grade using artificial intelligence, in which pre-trained artificial intelligence analyzes meat included in an image to measure taste and tenderness.

However, the technical problem to be achieved by the present embodiment is not limited to the technical problem described above, and other technical problems may exist.

### [Means for Solving the Problem]

As a technical means for achieving the above-described technical problem, an embodiment according to a first aspect of the present disclosure provides a method for measuring meat taste and tenderness using an artificial intelligence model. The method includes generating a training data set including at least one training meat image and a sensory evaluation table matched to the training meat image, and generating an artificial intelligence model trained to measure the taste and tenderness of meat included in an input meat image based on the training data set.

In addition, an embodiment according to a second aspect of the present disclosure provides an apparatus for measuring meat taste and tenderness. The apparatus includes a communication module, at least one processor, and a memory electrically connected to the processor and storing at least one code executed by the processor, wherein the memory stores code that, when executed by the processor, causes the processor to generate a training data set including at least one training meat image and a sensory evaluation table matched to the training meat image, and to generate an artificial intelligence model trained to measure the taste and tenderness of meat included in an input meat image based on the training data set.

### [Effects of the Invention]

The present invention can provide consistent and highly accurate measurement of meat taste and tenderness by artificial intelligence trained with meat images classified by taste and tenderness. In addition, since the measurement is performed by artificial intelligence, costs can be reduced and measurement speed can be improved.

Furthermore, by clarifying measurement criteria, it is possible to provide evidence in the form of visualized data indicating which factors played a decisive role in evaluating meat taste and tenderness.

### [BRIEF DESCRIPTION OF THE DRAWINGS]

FIG. 1 is a diagram illustrating a meat taste and tenderness measuring apparatus according to an embodiment of the present invention and a terminal communicatively connected thereto.

FIG. 2 is a diagram illustrating the meat taste and tenderness measuring apparatus of FIG. 1 in detail.

FIGS. 3A and 3B are diagrams illustrating a process of adjusting a meat image size and obtaining sensory evaluation according to an embodiment of the present invention.

FIG. 4 is a diagram illustrating a sensory evaluation result according to an embodiment of the present invention.

FIGS. 5A and 5B are diagrams illustrating a process of obtaining sensory evaluation according to an embodiment of the present invention.

FIGS. 6A and 6B are diagrams illustrating an interface provided to a terminal according to an embodiment of the present invention.

FIG. 7 is a flowchart illustrating a sequence of a method for measuring meat taste and tenderness according to another embodiment of the present invention.

### [DETAILED DESCRIPTION]

Hereinafter, the present disclosure will be described in detail with reference to the accompanying drawings. However, the present disclosure may be implemented in various different forms and is not limited to the embodiments described herein. In addition, the accompanying drawings are provided only to facilitate an easy understanding of the embodiments disclosed in the present specification, and the technical spirit disclosed in the present specification is not limited by the accompanying drawings. All terms, including technical and scientific terms, used herein should be interpreted as having meanings generally understood by those having ordinary skill in the art to which the present disclosure pertains. Terms defined in dictionaries should be interpreted as having meanings consistent with related technical literature and the presently disclosed content, and unless otherwise defined, should not be interpreted in an overly idealized or restrictive sense.

In order to clearly describe the present invention in the drawings, portions irrelevant to the description are omitted, and the size, shape, and form of each component illustrated in the drawings may be variously modified. Throughout the specification, the same or similar reference numerals are assigned to the same or similar components.

Throughout the specification, when a part is described as being “connected (coupled, contacted, or joined)” to another part, this includes not only a case in which the part is “directly connected (coupled, contacted, or joined)” but also a case in which the part is “indirectly connected (coupled, contacted, or joined)” with another member interposed therebetween. In addition, when a part is described as “including (comprising or provided with)” a component, this does not exclude other components unless otherwise specified, but means that other components may be further included (comprised or provided).

In the present specification, the term “unit” includes a unit implemented by hardware, a unit implemented by software, and a unit implemented using both hardware and software. In addition, one unit may be implemented using two or more pieces of hardware, and two or more units may be implemented using one piece of hardware. Meanwhile, the term “unit” is not limited to software or hardware, and may be configured to reside in an addressable storage medium or to reproduce one or more processors. Accordingly, as an example, a “unit” includes components such as software components, object-oriented software components, class components, and task components, as well as processes, functions, attributes, procedures, subroutines, segments of program code, drivers, firmware, microcode, circuitry, data, databases, data structures, tables, arrays, and variables. Functions provided within components and “units” may be combined into a smaller number of components and “units” or may be further separated into additional components and “units.” Furthermore, components and “units” may be implemented to reproduce one or more CPUs within a device or a secure multimedia card.

In the following description, suffixes such as “module” and “unit” assigned to components are used or mixed solely for convenience of description, and do not themselves have meanings or roles that distinguish them from each other. In addition, in describing the embodiments disclosed in the present specification, detailed descriptions of related known technologies are omitted when it is determined that such descriptions may obscure the gist of the embodiments disclosed herein.

Ordinal terms such as first and second used in the present specification are used only for the purpose of distinguishing one component from another component, and do not limit the order or relationship of the components. For example, a first component of the present disclosure may be named a second component, and similarly, a second component may also be named a first component. Singular expressions used in the present specification should be interpreted as including plural expressions unless clearly indicated otherwise.

The “user terminal” referred to hereinafter may be implemented as a computer or a portable terminal capable of accessing a server or another terminal through a network. Herein, the computer may include, for example, a notebook, desktop, or laptop equipped with a web browser, and a VR HMD such as HTC VIVE, Oculus Rift, GearVR, DayDream, or PSVR. The VR HMD includes PC-based devices such as HTC VIVE, Oculus Rift, FOVE, and Deepon, mobile-based devices such as GearVR, DayDream, Storm Mirror, and Google Cardboard, console-based devices such as PSVR, and independently implemented stand-alone models such as Deepon and PICO. The portable terminal may include, for example, a wireless communication device ensuring portability and mobility, such as a smartphone, a tablet PC, or a wearable device, as well as various devices equipped with communication modules such as Bluetooth Low Energy (BLE), NFC, RFID, ultrasonic, infrared, WiFi, and LiFi. In addition, the “network” refers to a connection structure capable of exchanging information among nodes such as terminals and servers, and includes a local area network (LAN), a wide area network (WAN), the Internet (World Wide Web), wired and wireless data communication networks, a telephone network, and wired and wireless television communication networks. Examples of the wireless data communication network include, but are not limited to, 3G, 4G, 5G, 3GPP, LTE, WiMAX, Wi-Fi, Bluetooth communication, infrared communication, ultrasonic communication, visible light communication (VLC), and LiFi.

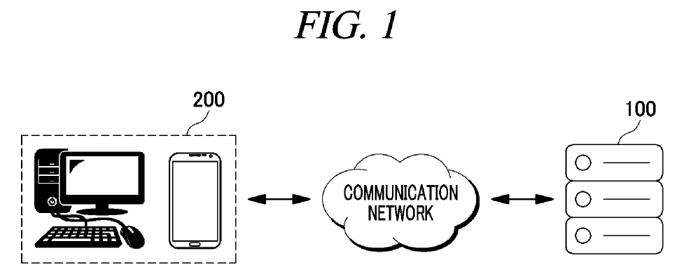

FIG. 1 is a diagram illustrating a meat taste and tenderness measuring apparatus according to an embodiment of the present invention and a terminal communicatively connected thereto.

Referring to FIG. 1, a meat taste and tenderness measuring apparatus (100) and a terminal (200) may be communicatively connected through a communication network. Hereinafter, the meat taste and tenderness measuring apparatus (100) may be referred to as a server. The server may be formed as a cloud computing server such as Software as a Service (SaaS), Platform as a Service (PaaS), or Infrastructure as a Service (IaaS). In addition, the server may be constructed in the form of a private cloud, a public cloud, or a hybrid cloud system, but the scope of the present invention is not limited thereto. Further, the meat taste and tenderness measuring apparatus (100) may be driven using an artificial intelligence model.

The meat taste and tenderness measuring apparatus (100) generates a training data set including at least one training meat image and a sensory evaluation table matched to the training meat image.

The meat taste and tenderness measuring apparatus (100) generates an artificial intelligence model trained to measure the taste and tenderness of meat included in an input meat image based on the training data set. Herein, taste may be determined based on quantitative marbling values, meat color values, moisture values, and tissue density values calculated based on evaluation values of multiple experts. In addition, tenderness refers to a degree of softness of meat, and may be determined based on marbling values, meat color values, moisture values, and tissue density values in the same manner as taste. In addition, according to an embodiment of the present invention, marbling values, meat color values, moisture values, and tissue density values may be determined for processed meat or aged meat to measure aging tenderness, a consumption period, or a post-processing period of meat.

The terminal (200) may transmit an image to the meat taste and tenderness measuring apparatus (100). In addition, the terminal (200) may receive meat taste and tenderness from the meat taste and tenderness measuring apparatus (100) in the form of visualized data.

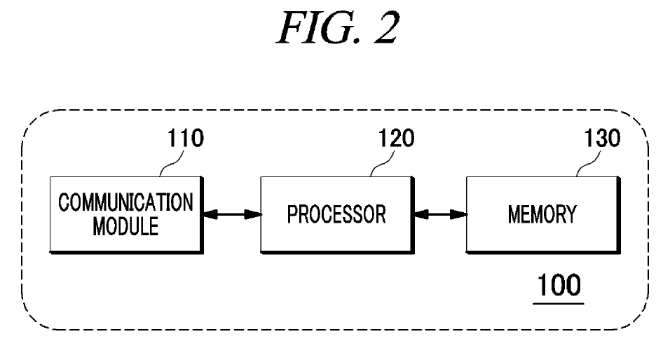

FIG. 2 is a diagram illustrating the meat taste and tenderness measuring apparatus of FIG. 1 in detail.

Referring to FIG. 2, the meat taste and tenderness measuring apparatus (100) may include a communication module (110), a processor (120), and a memory (130).

The communication module (110) may include a device including hardware and software necessary for transmitting and receiving signals such as control signals or data signals through wired or wireless connections with other network devices.

The communication module (110) may receive a meat image from the terminal (200). In addition, the communication module (110) may provide meat taste and tenderness to the terminal (200) in the form of visualized data including images, graphs, and tables. However, the present invention is not limited thereto, and as necessary, the communication module (110) may receive images or videos from an external device or a database other than the terminal (200).

The processor (120) may include various types of devices for controlling and processing data. The processor (120) may refer to a data processing device embedded in hardware and having physically structured circuits to perform functions expressed as codes or instructions included in a program.

As an example, the processor (120) may be implemented in the form of a microprocessor, a central processing unit (CPU), a processor core, a multiprocessor, an application-specific integrated circuit (ASIC), or a field programmable gate array (FPGA), but the scope of the present invention is not limited thereto.

The processor (120) performs operations according to code stored in the memory (130).

The memory (130) may store at least one of information and data input through the communication module (110), information and data required for functions performed by the processor (120), and data generated according to execution of the processor (120).

The memory (130) should be interpreted as collectively referring to non-volatile storage devices that continuously retain stored information even when power is not supplied and volatile storage devices that require power to retain stored information. The memory (130) may include, in addition to volatile storage devices requiring power to retain stored information, magnetic storage media or flash storage media, but the scope of the present invention is not limited thereto.

The memory (130) is electrically connected to the processor (120), and stores at least one code executed by the processor (120). The memory (130) stores code that causes the processor (120), when executed by the processor (120), to perform the following functions and procedures.

The memory (130) stores code that causes the processor (120) to generate a training data set including at least one training meat image and a sensory evaluation table matched to the training meat image. For example, the sensory evaluation table may include a marbling evaluation value, a meat color evaluation value, a texture evaluation value, a surface juiciness evaluation value, and a preference evaluation value for meat included in the meat image, and may be set based on criteria for determining taste and tenderness of beef or pork for a plurality of preset countries.

Herein, the marbling evaluation value may be calculated based on a fat distribution form of meat included in the meat image, the meat color evaluation value may be calculated based on saturation of the meat, the texture evaluation value may be calculated based on density, moisture, and deformability of the meat, the juiciness evaluation value may be calculated based on a fat content of the meat, and the preference evaluation value may be calculated based on a value preset by a subject who prepares the sensory evaluation table.

The memory (130) stores code that causes the processor (120) to generate an artificial intelligence model trained to measure taste and tenderness of meat included in an input meat image based on the training data set.

The memory (130) stores code that causes the processor (120) to receive a meat image that is a target of taste and tenderness measurement and to measure taste and tenderness of the meat image using the artificial intelligence model.

The memory (130) stores code that causes the processor (120) to generate visualized data in which a partial region of the meat image is visualized as a basis for the measured taste and tenderness. For example, the partial region may be a meat region in which a marbling distribution form and meat color included in the meat image are included at or above preset numerical values.

The memory (130) stores code that causes the processor (120) to provide, to the terminal (200), an interface including a meat image, visualized data, a marbling value, a meat color value, a texture value, a juiciness value, a total value, and a QR code.

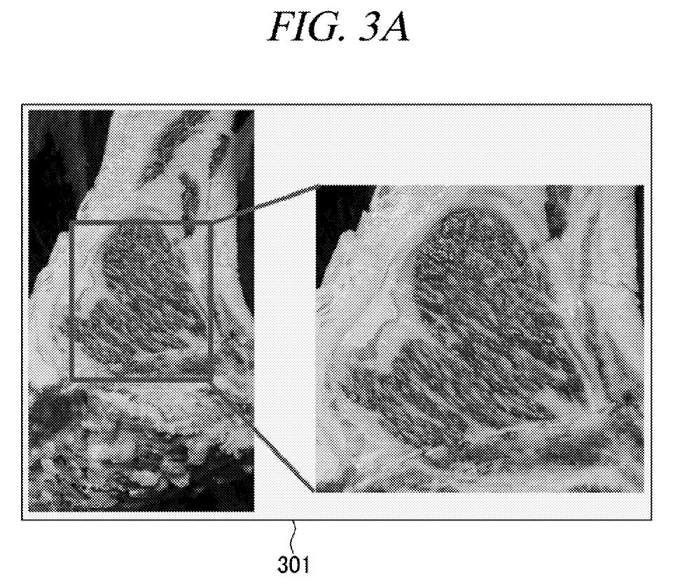

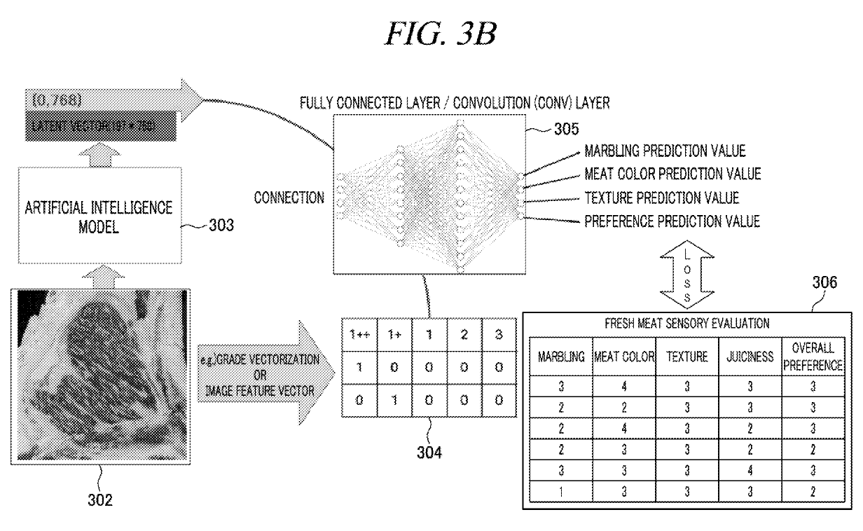

FIGS. 3A and 3B are diagrams illustrating a process of adjusting a meat image size and obtaining sensory evaluation according to an embodiment of the present invention.

Referring to FIG. 3A, the meat taste and tenderness measuring apparatus (100) may receive a meat image from a communicatively connected terminal (200), segment and crop a peripheral portion around meat from the meat image, and adjust a size of the image (resize) (301). Thereafter, the process illustrated in FIG. 3B may be performed. More specifically, the resized meat image (302) may be latent-vectorized (304) by an artificial intelligence model (303), taste and tenderness may be measured (305) based on pre-trained evaluation values of marbling, meat color, texture, juiciness, and preference for each grade, and a sensory evaluation table may be generated (306).

At this time, the artificial intelligence model may be a model for training, and may use a CNN (Convolutional Neural Network) and a ViT (Vision Transformer).

A CNN is a deep learning model that detects features of an image through convolution operations and, based thereon, classifies input data or extracts and learns meaningful information.

A ViT is a deep learning model that uses an attention mechanism for image processing, converts an image into tokens, and is effective in extracting and classifying global information.

More specifically, image tasks are implemented in a Transformer-structured model, and are implemented using a multi-head attention structure of a Transformer without using a CNN in vision tasks. Through this, the ViT exhibits excellent performance compared to other CNN-based models and can consume fewer computational resources during a training process.

At this time, large values are emphasized based on Q (Query), K (Key), and V (Value), which represent element expressions that are core to training.

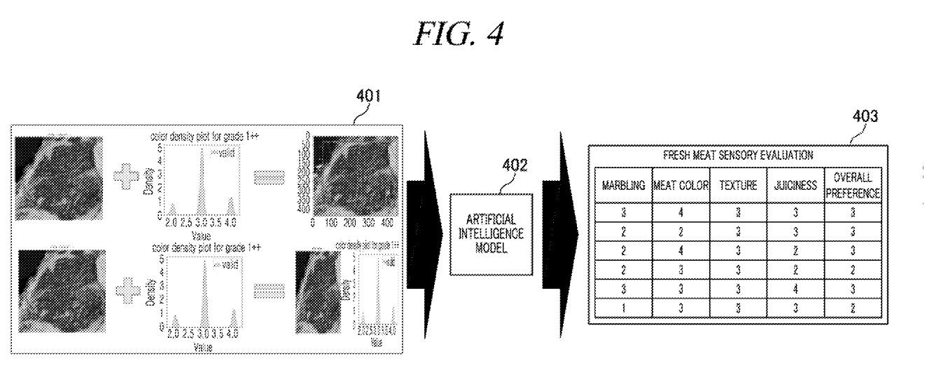

FIG. 4 is a diagram illustrating a sensory evaluation result according to an embodiment of the present invention.

Referring to FIG. 4, the meat taste and tenderness measuring apparatus (100) may receive a meat image from a communicatively connected terminal (200), and may combine and display a color distribution graph corresponding to a grade (401). At this time, the meat image and the color distribution graph may be arranged to overlap each other, or may be arranged side by side. In addition, a sensory evaluation result (403) may be obtained using an artificial intelligence model (402). At this time, the sensory evaluation result (403) may include marbling, meat color, texture, surface juiciness, and preference.

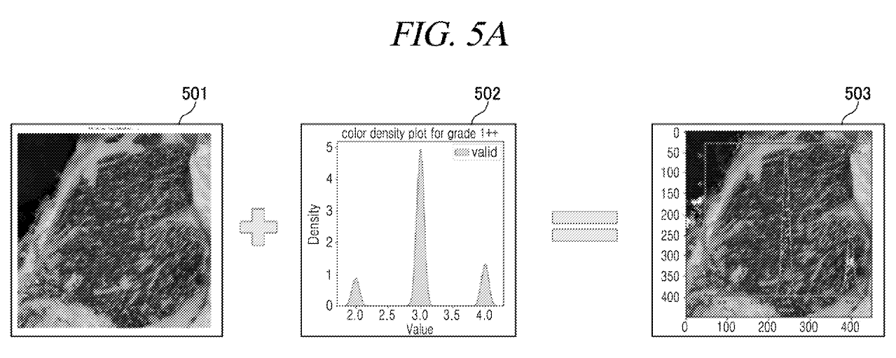

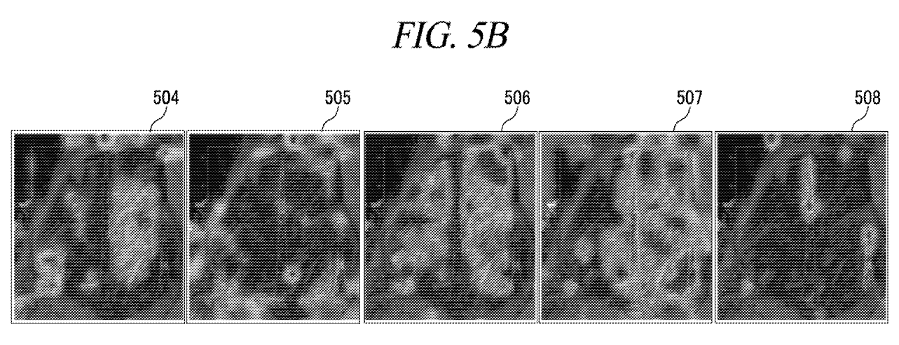

FIGS. 5A and 5B are diagrams illustrating a process of obtaining sensory evaluation according to an embodiment of the present invention.

Referring to FIG. 5A, as described with reference to FIG. 4, the meat taste and tenderness measuring apparatus (100) may receive a meat image from a communicatively connected terminal (200) (501), and may combine a color distribution graph corresponding to a grade (502) to display a color-distributed meat image (503). Thereafter, as illustrated in FIG. 5B, taste and tenderness of meat may be classified, and a result obtained by visualizing which portions contribute most by applying a Grad-CAM technique for each corresponding taste and tenderness of meat may be identified. In this regard, portions closer to red in the meat image indicate portions that contributed more to prediction by the model. For example, in FIG. 5B, taste and tenderness of meat are determined according to portions displayed in red in each of taste and tenderness distribution images (504 to 508).

At this time, Grad-CAM (Gradient-weighted Class Activation Mapping) is a technique for visualizing prediction results of a deep learning model, and can highlight and display how much a specific portion of an image contributes to a decision of the model. Through this, it is possible to visually understand which portions the model mainly utilizes, and to interpret predictions of the model. An attention map may be used together with Grad-CAM, and the attention map is a visualization technique in which a latent vector of a ViT (Vision Transformer)-based artificial intelligence model is reshaped into a square form and resized to the same size as an original image.

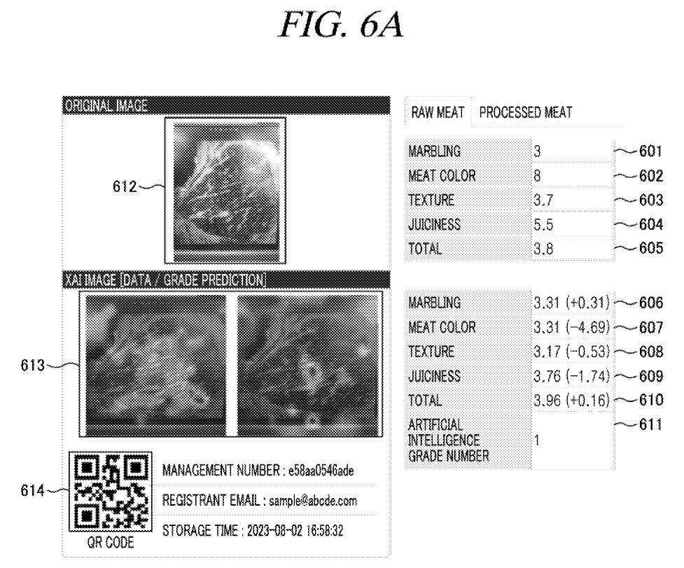

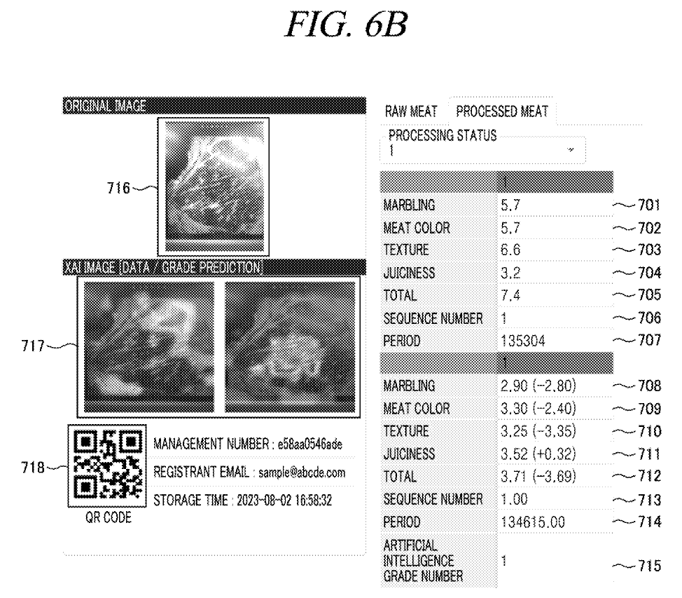

FIGS. 6A and 6B are diagrams illustrating an interface provided to a terminal according to an embodiment of the present invention.

Referring to FIG. 6A, a taste and tenderness evaluation result for raw meat (fresh meat) may provide marbling (601), meat color (602), texture (603), juiciness (604), and a total value (605) based on an original image (612), and may provide marbling (606), meat color (607), texture (608), juiciness (609), a total value (610), and an artificial intelligence grade number (611) based on an image (613) visualized by an artificial intelligence model. In addition, a QR code (614), a management number, a registrant email, and a storage time may be provided together.

Referring to FIG. 6B, a taste and tenderness evaluation result for processed meat may provide marbling (701), meat color (702), texture (703), juiciness (704), a total value (705), a sequence number (706), and a period (707) based on an original image (716), and may provide marbling (708), meat color (709), texture (710), juiciness (711), a total value (712), a sequence number (713), a period (714), and an artificial intelligence grade number (715) based on an image (717) visualized by an artificial intelligence model. In addition, a QR code (718), a management number, a registrant email, and a storage time may be provided together.

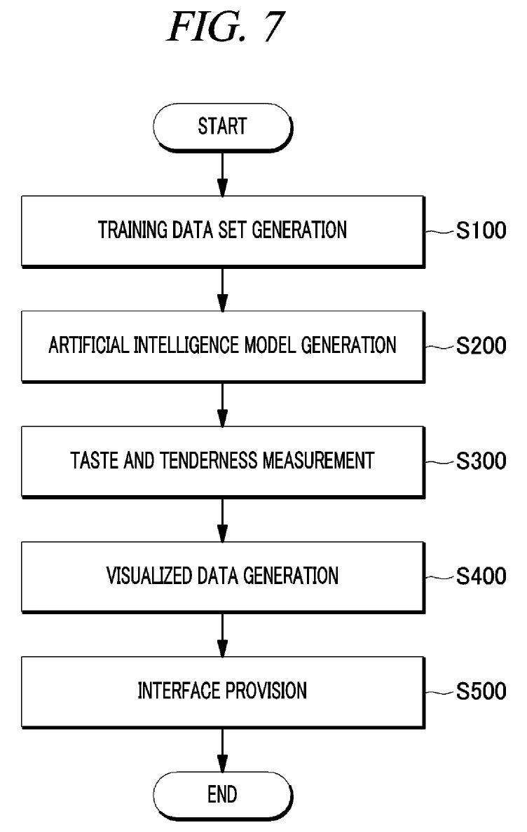

FIG. 7 is a flowchart illustrating a sequence of a method for measuring meat taste and tenderness according to another embodiment of the present invention.

Hereinafter, a meat taste and tenderness measuring method to be described may be performed by the meat taste and tenderness measuring apparatus (100) described above with reference to FIGS. 1 to 6B. Accordingly, descriptions of embodiments of the present disclosure described above with reference to FIGS. 1 to 6B may be equally applied to embodiments to be described below, and descriptions overlapping with those described above will be omitted. Steps described below are not necessarily performed in order, an order of the steps may be variously set, and the steps may be performed substantially simultaneously.

Referring to FIG. 7, the meat taste and tenderness measuring method includes a training data set generation step (S100), an artificial intelligence model generation step (S200), a taste and tenderness measurement step (S300), a visualized data generation step (S400), and an interface provision step (S500).

The training data set generation step (S100) is a step of generating a training data set including at least one training meat image and a sensory evaluation table matched to the training meat image.

The artificial intelligence model generation step (S200) is a step of generating an artificial intelligence model trained to measure taste and tenderness of meat included in an input meat image based on the training data set.

The taste and tenderness measurement step (S300) is a step of receiving a meat image that is a target of taste and tenderness measurement, and measuring taste and tenderness of the meat image using the artificial intelligence model.

The visualized data generation step (S400) is a step of generating visualized data in which a partial region of the meat image is visualized as a basis for the measured taste and tenderness.

The interface provision step (S500) is a step of providing, to the terminal (200), an interface including a meat image, visualized data, a marbling value, a meat color value, a texture value, a juiciness value, a total value, and a QR code.

An embodiment of the present invention may also be implemented in the form of a recording medium including computer-executable instructions such as program modules executed by a computer. A computer-readable medium may be any available medium that can be accessed by a computer, and includes both volatile and non-volatile media, and removable and non-removable media. In addition, the computer-readable medium may include all computer storage media. The computer storage media include volatile and non-volatile, removable and non-removable media implemented by any method or technology for storage of information such as computer-readable instructions, data structures, program modules, or other data.

Although the method and system of the present invention have been described with reference to specific embodiments, some or all of components or operations thereof may be implemented using a computer system having a general-purpose hardware architecture.

Those having ordinary skill in the art to which the present disclosure pertains will understand that various modifications and variations may be made without departing from the technical spirit or essential features of the present disclosure based on the above description. Accordingly, the embodiments described above should be understood as being illustrative in all respects and not restrictive. The scope of the present disclosure is defined by the appended claims rather than the foregoing detailed description, and all changes or modifications derived from the meaning and scope of the claims and equivalents thereof should be interpreted as being included within the scope of the present disclosure.

What is claimed is:

1. A method for measuring meat taste and tenderness using an artificial intelligence model, performed by a processor,
the method comprising:
a) generating a training data set including at least one training meat image and a sensory evaluation table matched to the training meat image; and
b) generating an artificial intelligence model trained to measure taste and tenderness of meat included in an input meat image based on the training data set.

2. The method of claim 1,
further comprising:
c) receiving a meat image that is a target of taste and tenderness measurement, and measuring taste and tenderness of the meat image using the artificial intelligence model.

3. The method of claim 1,
wherein the sensory evaluation table includes a marbling evaluation value, a meat color evaluation value, a texture evaluation value, a surface juiciness evaluation value, and a preference evaluation value for meat included in the meat image,
and is set based on criteria for determining taste and tenderness of beef or pork for a plurality of preset countries.

4. The method of claim 3,
wherein the marbling evaluation value is calculated based on a fat distribution form of meat included in the meat image,
the meat color evaluation value is calculated based on saturation of the meat,
the texture evaluation value is calculated based on density, moisture, and deformability of the meat,
the juiciness evaluation value is calculated based on a fat content of the meat,
and the preference evaluation value is a value preset by a subject who prepares the sensory evaluation table.

5. The method of claim 1,
further comprising:
d) generating visualized data in which a partial region of the meat image is visualized as a basis for the taste and tenderness measured according to step c).

6. The method of claim 5,
wherein the partial region is a meat region in which a marbling distribution form, meat color, texture, and juiciness included in the meat image are included at or above preset numerical values.

7. The method of claim 1,
further comprising:
e) providing, to a terminal, an interface including a meat image, visualized data, a marbling value, a meat color value, a texture value, a juiciness value, a total value, and a QR code.

8. An apparatus for measuring meat taste and tenderness, comprising:
a communication module;
at least one processor; and
a memory electrically connected to the processor and storing at least one code executed by the processor,
wherein the memory stores code that causes the processor, when executed by the processor,
to generate a training data set including at least one training meat image and a sensory evaluation table matched to the training meat image, and
to generate an artificial intelligence model trained to measure taste and tenderness of meat included in an input meat image based on the training data set.

9. The apparatus of claim 8,
wherein the memory stores code that causes the processor, when executed by the processor,
to receive a meat image that is a target of taste and tenderness measurement,
to measure taste and tenderness of the meat image using the artificial intelligence model,
to generate visualized data in which a partial region of the meat image is visualized as a basis for the measured taste and tenderness, and
to provide, to a terminal, an interface including a meat image, visualized data, a marbling value, a meat color value, a texture value, a juiciness value, a total value, and a QR code.

10. The apparatus of claim 8,
wherein the sensory evaluation table includes a marbling evaluation value, a meat color evaluation value, a texture evaluation value, a surface juiciness evaluation value, and a preference evaluation value for meat included in the meat image,
and is set based on criteria for determining taste and tenderness of beef or pork for a plurality of preset countries.

11. The apparatus of claim 10,
wherein the marbling evaluation value is calculated based on a fat distribution form of meat included in the meat image,
the meat color evaluation value is calculated based on saturation of the meat,
the texture evaluation value is calculated based on density, moisture, and deformability of the meat,
the juiciness evaluation value is calculated based on a fat content of the meat,
and the preference evaluation value is a value preset by a subject who prepares the sensory evaluation table.

12. The apparatus of claim 9,
wherein the partial region is a meat region in which a marbling distribution form, meat color, texture, and juiciness included in the meat image are included at or above preset numerical values.
